(19) United States
(12) Patent Application Publication
(10) Pub. No.: US 2022/0001062 A1
(43) Pub. Date: Jan. 6, 2022

(54) DISINFECTION OF AIR AND SURFACES WITH ULTRAVIOLET LIGHT

(71) Applicant: Microtek, Inc., San Diego, CA (US)

(72) Inventors: MyPhuong T. LE, San Diego, CA (US); Leah R. VILLEGAS, San Diego, CA (US); Tri Q. LE, San Diego, CA (US)

(73) Assignee: Microtek, Inc., San Diego, CA (US)

(21) Appl. No.: 17/366,341

(22) Filed: Jul. 2, 2021

Related U.S. Application Data
(60) Provisional application No. 63/106,411, filed on Oct. 28, 2020.
(60) Provisional application No. 63/047,619, filed on Jul. 2, 2020.

(51) Int. Cl. A61L 2/24; G01J 3/28; G01S 17/08; A61L 2/10; A61L 9/20

(52) U.S. Cl. CPC A61L 2/24; G01J 3/28; G01S 17/08; A61L 2/10; A61L 9/20; A61L 2209/111; A61L 2202/14; A61L 2202/122; A61L 2202/16; A61L 2209/12; A61L 2202/11

(57) ABSTRACT

Example systems and methods of UV disinfection of surfaces and/or air are presented which can utilize multiple peak wavelengths of UV light and utilize feedback control with UV light sensors. Some example systems and methods can facilitate sanitation of spaces which can be occupied by people and/or animals such as an interior room or space of a building or mode of transportation, or even an outdoor gathering space. Additionally, or alternatively, some example systems can include a compartment which is configured to block light exposure to people and/or animals during sanitation and into which objects can be placed for sanitation.

### CROSS REFERENCE TO RELATED APPLICATIONS

This application claims the benefit of priority to prior filed U.S. Provisional Patent Application No. 63/106,411 filed on Oct. 28, 2020 and prior filed U.S. Provisional Patent Application No. 63/047,619 filed Jul. 2, 2020, each of which is hereby incorporated by reference as set forth in full herein.

### FIELD OF INVENTION

The present invention generally relates to disinfection, and more particularly, to disinfection of pathogens in air and/or on surfaces using ultraviolet (UV) light.

### BACKGROUND

Transmission of infectious diseases is a global concern, highlighted by the rapid spread of the COVID-19 pandemic contemporary to the time of filing of the present disclosure. Infectious microorganisms in the air and on surfaces are major sources of disease transmission. There are several disinfection methods to help combat this issue, where chemicals such as ethanol, quaternary ammonium, and antibiotics, are commonly used. However, manual decontamination of surfaces using these chemicals is tedious, labor-intensive, expensive, and potentially toxic. Importantly, microbes are known to develop resistance mechanisms against these chemicals, evolving into “super bugs”, such as MRSA (Methicillin-Resistant Staphylococcus Aureus). Studies show that persistent contamination or recontamination is common even after thorough chemical cleaning.

Discovered in the late 1800s, ultraviolet germicidal irradiation (UVGI) is another disinfection approach that has recently become approved by the United States Center for Disease Control (CDC) as a method for the inactivation of a wide array of microbes. UVGI provides a technique for disinfecting microbes in the air, in water, and on surfaces. Most contemporary UVGI systems rely on wavelengths of UV light that are harmful to skin and eyes, making use of such systems difficult in practice. To limit harmful exposures to UV light, many systems block UV light with enclosures in which the UV light source is permanently or semi-permanently affixed (e.g. air treatment systems, disinfection boxes, etc.). Systems which allow free illumination of UV light rely primarily on users to exercise caution during installation and use (e.g. upper room air purifiers, hand-held UV lights, etc.) Further, because pathogens are invisible to the eye, it can be difficult to determine effectiveness of UVGI treatments. UVGI is effective when microorganisms are within the direct line-of-sight of the irradiation, thus producing inconsistent results for surfaces that are in shadowed areas or located far away from the UV source. As a result, the use of UVGI outside of controlled environments, like hospitals, is often haphazard, thus resulting in poor disinfectant effects and raising major safety concerns. Even within controlled environments, pathogens remain invisible to the eye, and therefore it can be difficult to validate effectiveness of UVGI and other disinfection systems.

### SUMMARY

Example systems and methods of UV disinfection of surfaces and/or air are presented which can utilize multiple peak wavelengths of UV light and utilize feedback control with UV light sensors. Some example systems and methods can facilitate sanitation of spaces which can be occupied by people and/or animals such as an interior room or space of a building or mode of transportation, or even an outdoor gathering space. Some example systems can include portable UV light fixtures when can be positioned to sanitize larger spaces that can be occupied by people or animals. Some example systems can include a compartment which is configured to block light exposure to people and/or animals during sanitation and into which objects can be placed for sanitation. Some example systems can include air circulation and disinfection.

An example ultraviolet (UV) disinfection and/or sterilization system can include at least one UV light detector and at least one UV light source in communication with the UV light detector(s). The UV disinfection and/or sterilization system can also include one or more of each of a user interface, a motion sensor, a compartment with a closable opening, an activation input, a sensor fixture, a portable light fixture, an event scheduling manager, a wireless transceiver, and/or luggage. The UV disinfection and/or sterilization system can be configured to disinfect surfaces and circulate air to disinfect the circulated air.

The UV light source(s) can be configured to illuminate with peak wavelengths of about 222 nm and about 254 nm and verify illumination at the peak wavelengths of about 222 nm and about 245 nm based at least in part on UV light detected by the UV light detector.

The UV light source can further be configured to cease illumination in response to detection, by the UV light detector(s), of a cumulative exposure to UV light over a predetermine threshold. When the UV disinfection and/or sterilization system includes multiple UV light detectors, each of the UV light detectors can be configured to detect a respective cumulative exposure to UV light. The UV light source can further be configured to cease illumination in response to each of the respective cumulative exposure to UV light reaching the predetermined threshold. As an alternative to relying on predetermined threshold(s) to cease illumination, the UV light detector can be configured to measure a first intensity of light at a wavelength of about 222 nm and a second intensity of light at a wavelength of about 254 nm, and the UV light source can cease illumination at a calculated time that is based at least in part on the first intensity of light and the second intensity of light.

The UV light detector can include a first UV light sensor and a second UV light sensor. The first UV light sensor can be configured to detect UV light within a first band of wavelengths which includes 222 nm and excludes 254 nm. The second UV light sensor can be configured to detect UV light within a second band of wavelengths which includes 254 nm and excludes 222 nm. The UV light detector can be configured to detect a cumulative exposure to UV light such that the cumulative exposure to UV light is based at least in part on UV light detected by the first UV light sensor in the first band of wavelengths and based at least in part on UV light detected by the second UV light sensor in the second band of wavelengths.

The user interface can be configured to communicate with the UV light source. The user interface can include an activation input. The UV light source can be configured to illuminate with peak wavelengths of about 222 nm and about 254 nm in response to a first activation of the activation input. The UV light source can further be configured to illuminate without the peak wavelength of about 254 nm in response to a second activation of the activation input.

The motion sensor can be configured to communicate with the UV light source. The UV light source can be configured to illuminate without the peak wavelength of about 254 nm in response to detection of motion by the motion sensor.

When the example includes a compartment, the closable opening of the compartment can be sized so that objects can pass through the opening when the opening is open. When the opening is closed, light can be inhibited from exiting the compartment through the opening. The light source and UV light detector are affixed within the compartment. The activation input can be configured to activate at least in part in response to closing of the closable opening. The UV light source can be configured to illuminate in response to activation of the activation input.

The UV light detector can be affixed to the sensor fixture. The UV light source can be affixed to the portable light fixture. The portable light fixture can be movable in relation to the sensor fixture. The portable light fixture and the sensor fixture can be configured to partially or automatically manage a disinfection operation in a location being disinfected by the UV disinfection system. The portable light fixture and the sensor fixture can be configured to transmit alert messages automatically configured based on one or more predetermined disinfection parameters of the location being disinfected. The alert messages can provide an indication of disinfection. The alert messages can be manually or automatically transmitted to a user computing device or other user computing devices securely connected therewith so that end-users can quickly and reliably detect that the location is disinfected. The sensor fixture can be configured to wirelessly communicate to the portable light fixture.

The event scheduling manager can be used to manually or automatically activate a disinfection operation by the UV disinfection system.

The wireless transceiver can be configured to receive a user input from a computing system separate and distinct from the UV light detector and the UV light source. The UV light source can be configured to illuminate in response to receiving the user input.

The luggage can be configured to transport the UV light detector and the UV light source. The luggage can be ruggedized or otherwise suitable for transporting scientific tools and/or electrical equipment.

The UV light source can include a distance sensor configured to measure a distance from a surface in line of sight of UV light from the UV light source.

Another example UV disinfection and/or sterilization system can include a UV light detector, a UV light source, a processor, and non-transitory computer readable medium. The UV disinfection and/or sterilization system can also include one or more of each of a user interface, a motion sensor, a compartment with a closable opening, an activation input, a sensor fixture, a portable light fixture, an event scheduling manager, a wireless transceiver, and/or luggage, each of which can be configured similarly to the corresponding components of the above example UV disinfection and/or sterilization system.

The UV light source can be configured to illuminate with peak wavelengths of about 222 nm and about 254 nm. The processor can be in communication the UV light detector and the UV light source. The non-transitory computer readable medium (referred to in this example as “memory” for the sake of brevity) can be in communication with the processor and can include instructions thereon, that when executed by the processor, cause the processor to perform various functions. The memory can include instructions to cause the processor to provide, in response to receiving an activation signal, a first control signal to the UV light source to cause the UV light source to illuminate. The memory can include instructions to cause the processor to receive a detector signal from the UV light detector. The memory can include instructions to cause the processor to verify, based at least in part on the detector signal, illumination of the UV light source at the peak wavelengths of 222 nm and 254 nm.

The memory can include instructions to cause the processor to determine, based at least in part on the detector signal, a cumulative exposure to UV light, the cumulative exposure to UV light to a predetermined threshold, and when the cumulative exposure to UV light is determined to be above the predetermined threshold as a result of the comparison, provide, a second control signal to the UV light source to cause the UV light source to cease illumination. The memory can further include instructions that control intensity of UV light output from the UV light source based at least in part on the detector signal. When the UV disinfection and/or sterilization system includes multiple UV light detectors, each of the UV light detectors can be configured to provide, to the processor, a respective detector signal. The memory can include instructions to cause the processor to receive the respective detector signals, determine a respective cumulative exposure to UV light for each of the respective detector signals, compare each of the respective cumulative exposure to UV light to the predetermined threshold, and when each of the respective cumulative exposure to UV light are determined to be above the predetermined threshold as a result of the comparison, provide, the second control signal to the UV light source to cause the UV light source to cease illumination.

As an alternative to ceasing illumination based on predetermined threshold(s), the memory can include instructions to cause the processor to determine, based at least in part on the detector signal, a first intensity of light at a wavelength of about 222 nm and a second intensity of light at a wavelength of about 254 nm, calculate, based at least in part on the first intensity and the second intensity, an exposure time, and provide, in response to elapse of the exposure time following the providing of the first control signal, a second control signal to the UV light source to cause the UV light source to cease illumination.

The user interface can be in communication with the processor and can be configured to provide the activation signal to the processor in response to a user input.

The memory can include instructions to cause the processor to receive a wavelength selection signal from the user interface and provide, based at least in part on the wavelength selection signal, a third control signal to the UV light source to cause the UV light source to illuminate without the peak wavelength of about 254 nm.

The memory can include instructions to cause the processor to wirelessly receive a user input from a computing device that is separate and distinct from the UV light detector and the UV light source and generate the activation signal based at least in part on the user input.

The UV light detector can include a first and second UV light sensor. The first UV light sensor can be configured to detect UV light within a first band of wavelengths including 222 nm and excluding 254 nm. The second UV light sensor can be configured to detect UV light within a second band of wavelengths including 254 nm and excluding 222 nm. The memory can include instructions to cause the processor to receive a first sensor signal from the first UV light sensor, receive a second sensor signal from the second UV light sensor, and calculate the cumulative exposure to UV light based at least in part on the first sensor signal and the second sensor signal.

The motion sensor can be configured to communicate with the processor. The memory can include instructions to cause the processor to receive a motion sense signal from the motion sensor and provide, based at least in part on the motion sense signal, a fourth control signal to the UV light source to cause the UV light to illuminate without the peak wavelength of about 254 nm.

The memory can include instructions to cause the processor to detect status of the closable opening of the compartment as open or closed, and when the status of the closable opening is open, provide a fifth control signal to the UV light source to inhibit the UV light source from illuminating at the peak wavelength of about 254 nm.

An example method of disinfection and/or sterilization can include one or more of the following steps executed in various orders as understood by a person skilled in the pertinent art according to the teachings herein. The method can include illuminating, by a UV disinfection and/or sterilization system, a UV light source of the UV disinfection system such that the UV light source illuminates with peak wavelengths of about 222 nm and about 254 nm. The method can include detecting, by the UV disinfection and/or sterilization system, illumination of light from the UV light source upon a UV light detector of the UV disinfection and/or sterilization system.

The method can include detecting, by the UV disinfection and/or sterilization system, a cumulative exposure of UV light incident upon the UV light detector. The method can include comparing, by the UV disinfection and/or sterilization system, the cumulative exposure of UV light to a predetermined threshold. When the cumulative exposure of UV light is determined to be above the predetermined threshold as a result of the comparison, the method can include ceasing, by the UV disinfection and/or sterilization system, illumination of the UV light source.

The method can include determining, by the UV disinfection and/or sterilization system, a first intensity of light at a wavelength of about 222 nm and a second intensity of light at a wavelength of about 254 nm incident upon the UV light detector when light from the UV light source is illuminated upon the UV light detector. The method can include calculating, by the UV disinfection and/or sterilization system, an exposure time based at least in part on the first intensity and the second intensity. The method can include ceasing, by the UV disinfection and/or sterilization system, illumination of the light from the UV light source upon the UV light detector in response to elapse of the exposure time following initial illumination of the light from the UV light source upon the UV light detector.

The method can include receiving a user input at a user interface of an ultraviolet (UV) disinfection system to cause the UV light source to illuminate.

The method can include receiving, by the user interface of the UV disinfection system, a wavelength selection input. The method can include illuminating, by the UV disinfection system, in response to receiving the wavelength selection input, the UV light source without the peak wavelength of about 254 nm. The method can include sensing, by a first UV light sensor of the UV light detector, UV light within a first band of wavelengths comprising 222 nm and excluding 254 nm and sensing, by a second UV light sensor of the UV light detector, UV light within a second band of wavelengths comprising 254 nm and excluding 222 nm. The method can include calculating, by the UV disinfection system, the cumulative exposure to UV light based at least in part on the UV light within the first band sensed by the first UV light sensor and UV light within the second band sensed by the second UV light sensor.

The method can include detecting motion by a motion sensor of the UV disinfection system. The method can include illuminating, by the UV disinfection system, in response to detecting motion, the UV light source without the peak wavelength of about 254 nm.

The method can include detecting, by a door sensor of the UV disinfection system, status of a door of the UV disinfection system as open or closed. When the status of the door is open, the method can include inhibiting, by the UV disinfection system, the UV light source from illuminating at the peak wavelength of about 254 nm.

The method can include configuring a portable light fixture to which the UV light source is affixed to be movable in relation to a sensor fixture to which the UV light detector is affixed.

The method can include wirelessly communicating from the sensor fixture to the portable light fixture. The method can include wirelessly receiving, at a user interface, user input transmitted from a computing device that is separate and distinct from the UV light detector and the UV light source and illuminating, by the UV disinfection system, the UV light source, in response to wirelessly receiving the user input.

The method can include transporting the UV light detector and the UV light source within luggage.

The method can include detecting, by the UV disinfection system, a respective cumulative exposure of UV light incident upon a plurality of UV light detectors of the UV disinfection system, the plurality of UV light detectors comprising the UV light detector. The method can include comparing, by the UV disinfection system, respective cumulative exposure to UV light for each of the plurality of UV light detectors to the predetermined threshold. When the cumulative exposure to UV light for each of the plurality of UV light detectors is determined to be above the predetermined threshold, the method can include ceasing, by the UV disinfection system, illumination of the UV light source.

The method can include measuring, by a measurement sensor of the UV light source, a distance from the UV light source to a surface in line of sight of UV light from the UV light source.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example UV disinfection system according to aspects of the present invention;

FIG. 2 is an illustration of another example UV disinfection system according to aspects of the present invention;

FIG. 3A is an illustration of another example UV disinfection system including a portable UV light source according to aspects of the present invention;

FIG. 3B is an illustration of example UV light emitters of an example UV light source according to aspects of the present invention;

FIG. 3C is an illustration of an example rechargeable battery usable with an example UV disinfection system according to aspects of the present invention;

FIG. 4 is an illustration of another example UV disinfection system configured to disinfect a space which is sized to be occupied by people and/or animals according to aspects of the present invention;

FIG. 5 is an illustration of another example UV disinfection system, the illustrated system including disinfection compartments according to aspects of the present invention;

FIG. 6 is an illustration of another example UV disinfection system, the illustrated system including modular disinfection compartments according to aspects of the present invention;

FIGS. 7A through 7C are flow diagrams illustrating methods of a decontamination process according to aspects of the present invention; and

FIGS. 8A and 8B are spectrographs illustrating spectral output of an example UV light source in different modes of operation according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g. “about 90%” may refer to the range of values from 71% to 99%.

As used herein, the term “computing system” is intended to include standalone machines or devices and/or a combination of machines, components, modules, systems, servers, processors, memory, detectors, user interfaces, computing device interfaces, network interfaces, hardware elements, software elements, firmware elements, and other computer-related units. By way of example, but not limitation, a computing system can include one or more of a general-purpose computer, a special-purpose computer, a processor, a portable electronic device (e.g. tablet, phone, etc.), a portable electronic medical instrument, a stationary or semi-stationary electronic medical instrument, or other electronic data processing apparatus.

As used herein, the terms “disinfection” and “sterilization” are each intended to describe decontamination processes. As used herein, “disinfection” is a process of killing at least a portion of harmful microorganisms, but not all microorganisms of a target. As used herein, “sterilization” is a process of killing all microorganisms of a target. Example systems and methods disclosed herein can be configured to achieve “disinfection” or “sterilization” depending on specific implementation of the example systems and methods. Unless specifically stated or otherwise readily apparent to a person skilled in the pertinent art, an example system or method described herein in terms “disinfection” does not preclude the possibility that the example system or method can be used to perform “sterilization”. For ease of discussion, the terms “disinfection”, “disinfect”, etc. are used predominantly herein, and this usage is not to be construed as precluding “sterilization”. Likewise, unless specifically stated or otherwise readily apparent to a person skilled in the pertinent art, an example system or method described herein in terms of “sterilization” does not preclude the possibility that the example system or method can be used to perform “disinfection”.

As used herein, the term “non-transitory computer-readable media” includes, but is not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

As used here, the term “processor” is intended to include one or more processing units (e.g., in a multi-core configuration). Further, processors described herein can be implemented using one or more heterogeneous processor systems in which a main processor is present with secondary processors on a single chip. As another illustrative example, the processors can be a symmetric multi-processor system containing multiple processors of the same type. Further, the processors can be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein. Additionally, the processors may perform partial processing and receive partial processing by a processor and/or computing device communicatively coupled to the processors.

As used herein, the terms “ultraviolet light”, “UV light”, “ultraviolet”, and “UV” are intended to include electromagnetic radiation with a wavelength from about 10 nanometers (nm) to about 400 nm, shorter than visible light, longer than X-rays, and including UVA (315 nm-400 nm), UVB (280 nm-315 nm) and UVC (100 nm-280 nm). Unless specified otherwise or made apparent to a person skilled in the pertinent art in context, UV light can originate from a natural and/or man-made source.

As used herein, the term “UV light detector” is intended to include an apparatus including a “dosimeter”, which is a device used to measure an absorbed dose of ionizing radiation.

Example UVGI systems and methods are presented herein that can employ several smart technologies, including, but not limited to integrated sensors and/or wireless app control. Specific features the example UVGI systems and methods, either alone or in combination, can enable functionality and processes that allow for monitoring and optimization of UV doses for effective and safe disinfection and/or sterilization.

Table 1 lists some microbes including viruses, bacteria, and molds related to illness. As indicated in Table 1, far-UVC (222 nm) can be just as efficient at disinfecting some microbes as UV-C (254 nm). Microbes that can be disinfected by far-UVC can include viruses (e.g. Escherichia virus MS2, H1N1, HCoV-229E, HCoV-OC43), bacteria (e.g. Clostridium difficile spore, Escherichia coli, MRSA, Pseudomonas aerginosa, Salmonella typhimurim), and molds and yeast (e.g. Aspergillus niger spore, Candida albicans) as summarized in Table 1. Although no data is reported related to SARS-CoV, subsequent studies may show that 222 nm is effective at disinfecting SARS-CoV as well as additional microbes not listed. Future studies may also reveal other wavelengths of light that can be effective to disinfect some microbes. In the table, four asterisks (****) indicate UV dose to achieve 3-log reduction in microbe is less than 50 millijoules per centimeter cubed (mJ/cm³) or a treatment with 70% ethanol for less than 10 seconds to achieve 3-log reduction of the microbe; three asterisks (***) indicate UV dose to achieve 3-log reduction in microbe is approximately 100 mJ/cm³ or a treatment with 70% ethanol for approximately 20 seconds to achieve 3-log reduction of the microbe; two asterisks (**) indicate UV dose to achieve 3-log reduction in microbe is approximately 1000 mJ/cm³ or a treatment with 70% ethanol for approximately 30 seconds to achieve 3-log reduction of the microbe; one asterisk (*) indicates a treatment with 70% ethanol for greater than 30 seconds to achieve 3-log reduction of the microbe; and N.D. indicates no data.

TABLE 1

| | METHODS | | |
|---|---|---|---|
| | Far-UVC | UV-C | 70% |
| Microbe | (222 nm) | (254 nm) | Ethanol |

Viruses

| | | | |
|---|---|---|---|
| Escherichia virus MS2 | **** | **** | N.D. |
| H1N1 (swine flu) | **** | **** | * |
| Human coronavirus alpha | **** | N.D. | N.D. |
| (HCoV-229E) | | | |
| Human coronavirus beta | **** | N.D. | N.D. |
| (HCoV-OC43) | | | |
| SARS-CoV | N.D | **** | **** |

Bacteria

| | | | |
|---|---|---|---|
| Clostridium difficile: spore | **** | *** | * |
| Escherichia coli | **** | **** | **** |
| Methicillin-resistant | **** | **** | **** |
| Staphylococcus aureus (MRSA) | | | |
| Pseudomonas aeruginosa | **** | **** | **** |
| Salmonella typhimurium | **** | **** | **** |

Molds and Yeast

| | | | |
|---|---|---|---|
| Aspergillus niger: spore | ** | ** | **** |
| Candida albicans | **** | **** | **** |

Table 2 includes a comparison of safety of far-UVC (222 nm), UV-C (254 mm), and disinfecting chemicals with effects considered on skin, eyes, and lungs. Exposure to 222 nm wavelength UV light at dosages effective at disinfecting microbes may likely be safe for the skin and eyes. Far-UVC (222 nm) likely does not penetrate mammalian cells and likely does not damage Deoxyriboneucleic Acid (DNA) to the same degree as UV-C. Future studies may also reveal other wavelengths of light that can be effective to disinfect some microbes while also presenting reduced risk of adverse health effects compared to 254 nm UV light.

TABLE 2

| | METHODS | | |
|---|---|---|---|
| | Far-UVC | UV-C | |
| Tissue | (222 nm) | (254 nm) | 70% Ethanol |
| Skin | YES | NO | NO |
| Eyes | YES | NO | NO |
| Lungs | N.D. | N.D. | NO |

Table 3 illustrates potential synergistic effects of simultaneously dosing with 222 nm and 254 nm wavelengths. Modality facilitating the synergy is believed to include inducing reactive oxygen species production and inactivation of reactive oxygen species defense mechanisms in microbes including E. coli, S. Typhimurium, and L. monocytogenes as summarized in Table 3. Future studies may also reveal other combinations of wavelengths of light that can be effective to disinfect some microbes with a synergistic effect. In Table 3, for E. Coli Far-UV (222 nm) was applied at 1.05 mJ/cm³ and UV-C (254 nm) was applied at 1.10 mJ/cm³; and for S. Typhimurium and L. monocytogenes Far-UV (222 nm) was applied at 3.15 mJ/cm³ and UV-C (254 nm) was applied at 3.30 mJ/cm³. Log reduction follows the formula: [log₁₀(N₀/N)] where No is the initial population and N is the population after treatment.

TABLE 3

| | METHODS | | |
|---|---|---|---|
| | Far-UVC | UV-C | |
| Microbe | (222 nm) | (254 nm) | 70% Ethanol |
| E. coli O157:H7 | 3.30 ± 0.65 | 1.71 ± 0.09 | 7.59 ± 0.04 |
| S. Typhimurium | 3.58 ± 0.24 | 1.98 ± 0.57 | 6.82 ± 0.16 |
| L. monocytogenes | 2.07 ± 0.06 | 2.78 ± 0.40 | 6.90 ± 0.23 |

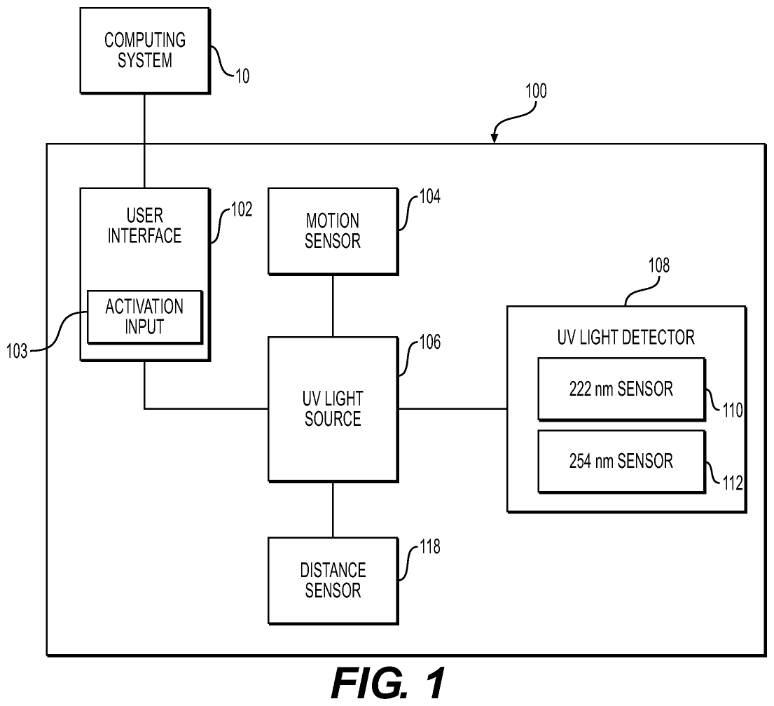

FIG. 1 is an illustration of an example UV disinfection system 100. The system 100 is illustrated as including a user interface 102, a motion sensor 104, a UV light source 106, a UV light detector 108, and a distance sensor 118. The motion sensor 104 can be optional in some cases depending on the likelihood of exposure of people and/or animals to harmful dosages of UV light. The distance sensor 118 can be optional and can be used to determine distance between the UV light source 106 and surfaces to be disinfected as needed or desired. As illustrated, the UV light source 106 can be configured to communicate with the user interface 102, motion sensor 104, UV light detector 108, and distance sensor 118. The UV light source 106 can provide UV illumination based on communications with the user interface 102, motion sensor 104, UV light detector 108, and distance sensor 118. In some examples the system 100 can be assembled as a single apparatus or device. Alternatively, portions of the apparatus can be movable in relation to others, e.g. the UV light source 106 and UV light detector 108 can be movable independent of each other.

As illustrated in FIG. 1, the UV light source 106 is centric to the system 100. Alternatively, functionality as described herein can be carried out in other arrangements as understood by a person skilled in the pertinent art according to the teachings herein. For instance, the system 100 can be configured with the user interface 102 being centric such that the user interface 102 is configured to receive signals from the motion sensor 104 and UV light detector 108 to provide commands to the UV light source 106. In such an example, computations and signal processing for controlling the UV light source 106, which are based signals from the motion sensor 104 and UV light detector 108, can be carried out at the user interface 102 and/or can be carried out by an external computing system 10 in communication with the user interface 102.

The external computing system 10 can be any suitable computing system configured to communicate with the UV disinfection system 100. For instance, the external computing system can include a general purpose computing device such as tablet, phone, or laptop running an application to cause the tablet, phone, or laptop to communicate with the user interface 102. From the perspective of functionality, such an application can be considered a part of the UV disinfection system 100.

In some examples, the user interface 102 can be configured to control the UV disinfection system 100 without requiring the external computing system 10. In some examples, the user interface 102 can be relatively minimalistic such that several functions described herein related to the UV disinfection system 100 can be carried out autonomously, without requiring user input.

The user interface 102 can include an activation input 103. The UV light source 106 can be configured to illuminate in response to an activation of the activation input 103. The activation input 103 can include a button (e.g. “on button”), a sensor which activates when a user interacts with the system 100 (e.g. user closes a door), a wireless receiver or transceiver configured to receive an activation signal from an external device (e.g. the external computing system 10), other suitable activation device as understood by a person skilled in the pertinent art, and any combination thereof. In some examples, the user interface 102 can include a wireless transceiver and additional hardware and software to facilitate communications between the UV light source 106 and the external computing system 10 so that a user need not interact directly with the user interface 102, but rather directly with the computing system 10, to control the UV light source 106. Additionally, or alternatively, the user interface 102 can include buttons, displays, touch screens, and/or other user input mechanisms to allow the user to directly interact with the user interface 102.

The UV light source 106 can be configured to illuminate with peak wavelengths of about 222 nanometers (nm) and 254 nm in response to activation of the activation input 103. Additionally, or alternatively, the UV light source 106 can be configured to illuminate with peak wavelengths, or wavelength bands, effective to disinfect microbes such as the microbes listed in Table 1 and Table 3 and otherwise described herein. The UV light source 106 can be configured to illuminate with peak wavelengths in the UVA, UVB, and/or UVC spectrums; each of these spectrums have been shown to have germicidal effects. In addition to, or as an alternative to providing UV light, the light source 106 can be configured to illuminate with peak wavelengths in the near infrared (˜700 nm) and/or radio frequency (˜1 m, 300 MHz) spectrums; each of these spectrums have been shown to have germicidal effects.

The UV light detector 108 can be positioned so that light illuminating from the UV light source 106 is incident upon sensors 110, 112 of the detector 108. The UV light detector 108 can include a 222 nm sensor 110 configured to detect UV light within a band of wavelengths that includes 222 nm and excludes 254 nm. The UV light detector 108 can include a 254 nm sensor 112 configured to detect UV light within a band of wavelengths that includes 254 nm and excludes 222 nm. Additionally, or alternatively, the UV light detector 108 can include sensors configured to detect UV light within other bands of wavelengths.

Preferably, but not necessarily, the UV light detector 108 is configured to detect wavelengths of UV light which can be output by the UV light source 106. For instance, the UV light detector 108 can include a UV light detector to detect exposure to a single wavelength of light and the system 100 can be configured to calculate exposure of additional wavelengths of light output from the UV light source 106 based on detected exposure of the single wavelength of light a known relative power provided to various lamps of the UV light source 106 illuminating at the other wavelengths. In some examples, the UV light detector 108 can include sensors configured to detect UV light that is not provided by the UV light source 106 (e.g. UV light present in sunlight or other light sources).

Cumulative exposure to UV light (i.e. “dose” of UVGI treatment) incident upon the UV light detector 108 can be calculated or otherwise determined based on signals from one or both of the sensors 110, 112. For instance, UV intensity can be monitored at each of the sensors 110, 112, and the intensity can be used to calculate exposure time to achieve a desired dose. The UV light source can be configured to cease illumination following elapse of the exposure time. Alternatively, an exposure time need not be calculated and the UV light source 106 can be configured to cease illumination when the cumulative exposure to UV light reaches a predetermined threshold. The predetermined threshold can be based on dosages effective to disinfect one or more target microbes. As another alternative, exposure time can be calculated, predetermined threshold of exposure can be determined, the UV light source can be configured to cease illumination either following the elapse of exposure time or when the cumulative exposure to UV light reaches a predetermined threshold. At completion of illumination actual dosage can be compared to the predetermined threshold or actual exposure time can be compared to the calculated exposure time as a check for proper operation during the illumination.

In some examples, the distance sensor 118 can be used by the system 100 to determine a relative position of the UV light source 106 from a surface to be disinfected. The distance sensor can include LiDAR or other such sensor as understood by a person skilled in the pertinent art. The system 100 can calculate an effective dose based on the relative position of the UV light source 106 to the surface. The predetermined threshold and/or calculated exposure time can be determined based on the effective dose. Intensity of light output during exposure can also be adjusted based on the relative position of the UV light source 106 from the surface to be disinfected, target microbe, desired degree of disinfection/sterilization, and/or desired exposure time. In some examples, the relative position of the UV light source 106 and UV light detector 108 can be determined by the system 100. In such examples, the system 100 can be configured to determine the predetermined threshold and/or calculated exposure time based on the relative position of the UV light detector 108 or the UV light source 106.

Exposure to UV light required to disinfect several microbes of interest are known, and work is ongoing to determine effective dosages for additional microbes. Effective dosage can differ depending on the wavelength peak(s) present in the UV light. For instance, a dose of 50 millijoules per centimeter squared (mJ/cm²), 100 mJ/cm², or 1,000 mJ/cm² of UV light at 222 nm or 254 nm wavelengths, applied separately, can be effective at achieving at least a 3 log reduction of several microbes as indicated in Table 1. For some microbes, including those listed in Table 3, a treatment combining simultaneous doses of UV light at 222 nm and UV light at 254 nm can result in a log reduction in microbes greater than the sum of log reduction of microbes when does of UV light at 222 nm and UV light at 254 nm are applied separately. Future work may reveal other combinations of UV (and potentially non-UV) light wavelengths with a synergistic effect. When such combinations are discovered, the UV light source 106 and UV light detector 108 can be modified to utilize such combinations of UV light as understood by a person skilled in the pertinent art according to the teachings herein.

Because of the synergistic effect of simultaneous dosage with UV light 222 nm and 254 nm, disinfection can be achieved more quickly and/or be more effective (kill greater quantity and/or variety of microbes) for a give disinfection time when the UV light source 106 is configured to provide both wavelengths at a given light intensity compared to providing only one wavelength at the same given intensity. However, because UV light at 254 nm can be harmful to skin and eyes, in some circumstances it may be necessary to deactivate the 254 nm UV light output. In those circumstances, it may be possible to continue to provide 222 nm UV light output because the UV light at 222 nm may be able to be applied at a dosage effective to disinfect certain microbes while posing an acceptably low risk of injury to skin and eyes. To that end, the system 100 can include inputs to cause the UV light source to illuminate with the 222 nm UV light and without the 254 nm UV light. The motion sensor 104 can be positioned and otherwise configured to detect motion which may indicate that a person and/or animal is at risk of being exposed to a harmful dosage of UV light at 254 nm. The UV light source 106 can be configured to cease illumination at 254 nm and continue illumination at 222 nm in response to detection of motion by the motion sensor 104. The motion sensor 104 can be realized with hardware and/or software as understood to a person skilled in the pertinent art, including but not limited to LiDAR, infrared sensors, microwave sensors, ultrasonic sensors, vibration sensors, contact sensors (e.g. on entryways), video sensors, etc. Additionally, or alternatively, a user can provide an input to the user interface 102 to cause the UV light source 106 to cease illumination at 254 nm and continue illumination at 222 nm.

The UV disinfection system 100 need not specifically be configured to illuminate at peak wavelengths of 222 nm and 254 nm and can alternatively be configured, in a similar manner as understood by a person skilled in the pertinent art according to the teachings herein, with two or more light sources that have a similar synergistic effect and differing levels of risk of exposure.

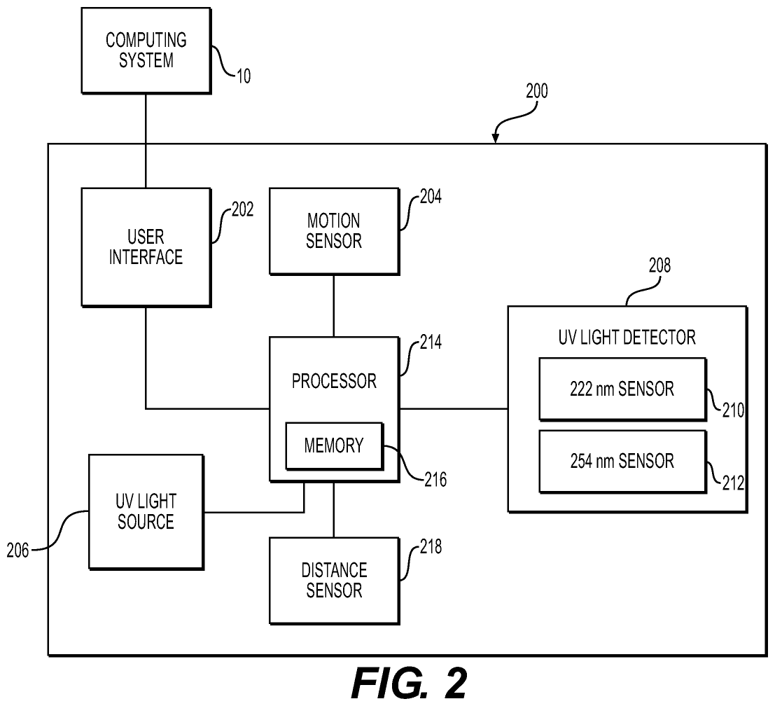

FIG. 2 illustrates another example UV disinfection system 200 having a user interface 202, motion sensor 204, UV light source 206, a UV light detector 208, and distance sensor 218 with similar functionality and configurations as the corresponding components 102, 104, 106, 108, 118 illustrated in FIG. 1. FIG. 2 illustrates the system 200 can include a central processor 214 and memory (non-transitory computer readable medium) 216 in communication with the processor (integral to the processor as illustrated) with instructions thereon that can be executed by the processor 214. In some examples the system 200 can be assembled as a single apparatus or device. Alternatively, portions of the apparatus can be movable in relation to others, e.g. the UV light source 206 and UV light detector 208 can be movable independent of each other in some examples. In example systems where portions of the apparatus are movable in relation to each other, the processor 214 can be movable together with the user interface 202, UV light source 206, UV light detector 208, or motion sensor 204 (e.g. by virtue of being affixed to a common housing). Further, in example systems where portions of the apparatus are movable in relation to each other, each separable portion can include a processor and memory such that the combined processors and memory of the separable portions together provide functionality of the processor 214 and memory 216 as described in relation to FIG. 2.

The processor can be configured to receive an activation signal from the user interface 202. The activation signal can be an electrical signal received an input pin of the processor 214. The processor 214 can be configured to receive the activation signal directly from the user interface 202 or indirectly via a wired or wireless communication with the user interface 202 (e.g. via transmission through transceivers, analog-to-digital converters, etc.). The user interface 202 can include an activation input similar to the activation input 103 illustrated in FIG. 1. The user interface 202 can be configured to communicate with an external computing system 10 to provide indirect interaction with the system 200 and/or external computing similar to as described in relation to the user interface 102 illustrated in FIG. 1. The memory 216 can include instructions thereon to cause the processor 214 to recognize when the activation signal is received.

The UV light source 206 can be configured to illuminate with peak wavelengths of 222 nm and 254 nm and/or other wavelengths and wavelength bands effective to disinfect microbes similar to the UV light source 106 illustrated in FIG. 1. In some examples, one or more target microbes and degree of disinfection/sterilization can be selected via the user interface 202, and a predetermined threshold and/or exposure time can be determined or calculated based at least in part on the selected target microbes and degree of disinfection/sterilization. Upon receiving the activation signal from the user interface 202, the processor 214 can be configured to provide a control signal to the UV light source 206 to cause the UV light source to illuminate. The memory 216 can include instructions that when executed by the processor 214, cause the processor to provide the control signal to the UV light source 206 upon receiving the activation signal from the user interface 202.

The UV light detector 208 can be configured with similar functionality as the UV light detector 108 illustrated in FIG. 1. The UV light detector 208 can include a 222 nm sensor 210 and a 254 nm sensor 212 functioning similarly to the corresponding sensors 110, 121 illustrated in FIG. 1. The processor 214 can be configured to receive a detector signal from the UV light detector 208. The detector signal can be an electrical signal input to an input or pin of the processor 214. The processor 214 can be configured to receive the detector signal directly from the UV light detector 208 or indirectly via a wired or wireless communication with the UV light detector 208 (e.g. via transmission through transceivers, analog-to-digital converters, etc.). The memory 216 can include instructions thereon to cause the processor 214 to receive the detector signal.

The processor 214 can be configured to receive a first sensor signal, directly or indirectly, from the 222 nm sensor 210. The processor 214 can be configured to receive a second sensor signal, directly or indirectly, from the 254 nm sensor 212. The memory 216 can include instructions thereon that can be executed by the processor 214 to verify that UV light at 222 nm and 254 nm is illuminating from the UV light source 206 upon the UV light detector 208.

The system 200 can be configured to determine and provide a desired dosage through various alternative methods.

In a first example, the memory 216 can include instructions thereon that can be executed by the processor 214 to perform calculations and processes to determine the cumulative exposure to UV light. The memory 216 can include instructions thereon to cause the processor 214 to receive the first and second sensor signals and calculate the cumulative exposure to UV light based at least in part on the first and second sensor signals. The processor 214 can be configured to compare the cumulative exposure to UV light to a predetermined threshold. The predetermined threshold can be based on dosages of UV light effective to disinfect one or more target microbes. The memory 216 can include instructions including the predetermined threshold (e.g. as a program variable). The memory 216 can include instructions to cause the processor 214 to compare the cumulative exposure to UV light to the predetermined threshold. The memory 216 can include instructions to cause the processor 214 to provide a control signal to the UV light source 206 to cease illumination when the cumulative exposure to UV light is determined to be able the predetermined threshold.

In a second example, the memory 216 can include instructions thereon that can be executed by the processor 214 to determine, based at least in part sensor signals from the UV light detector 208, a first intensity of light at a wavelength of about 222 nm and a second intensity of light at a wavelength of about 254 nm when the UV light source 206 is illuminating upon the UV light detector 208; calculate an exposure time based at least in part on the first intensity and the second intensity; and provide a control signal to the UV light source 206 to cause the UV light source to cease illumination following elapse of the calculated exposure time.

In a third example, the predetermined threshold and exposure time can be calculated as in the first and second examples, illumination of the UV light source 206 can cease based on the predetermined threshold as in the first example or based on the calculated exposure time as in the second example, and the memory 216 can include instructions thereon that cause be executed by the processor 214 to either (i) compare an actual exposure time to the calculated exposure time if illumination of the UV light source 206 is ceased according to the first example; or (ii) compared an actual dosage at the UV detector 208 to the predetermined threshold if illumination of the UV light source 206 is ceased according to the second example. In either case (i) or (ii), the system 200 can be configured to provide an indication that operation of the system 200 during illumination of the UV light source 206 functioned as expected (or not).

The processor 214 can be configured to activate the distance sensor 218 to determine a relative position of the UV light source 206 to a target surface to be disinfected. The processor 214 can be configured to determine the predetermined threshold and/or calculate exposure time based at least in part on the relative position of the UV light source 206 and the target surface. The processor 214 can further be configured to determine the relative position of the UV light source 206 to the UV light detector 208. The processor 214 can be configured to determine the predetermined threshold and/or calculate exposure time based at least in part on the relative position of the UV light source 206 and the UV light detector 208. The memory 216 can include instructions thereon to facilitate interactions between the processor 214 and the distance sensor 218 and resulting actions of the system 100 based on those interactions.

In some applications it may be desirable to deactivate the 254 nm light output. The processor 214 can be configured to receive, directly or indirectly, a wavelength selection signal from the user interface 202. The wavelength selection signal can be generated based on a user input at the user interface 202 or at the computing system 10 whereby the user chooses to deactivate the 254 nm light output. The memory 216 can include instructions to cause the processor 214 to recognize receipt of the wavelength selection signal and, in response, provide a control signal to the UV light source to cause the UV light source to illuminate without the 254 nm output.

The processor 214 can be configured to receive, directly or indirectly, a motion sense signal from the motion sensor 204. The memory 216 can include instructions to cause the processor 214 to recognize receipt of the motion sense signal and provide a control signal to the UV light source to cause the UV light source to cease illumination of the 254 nm output.

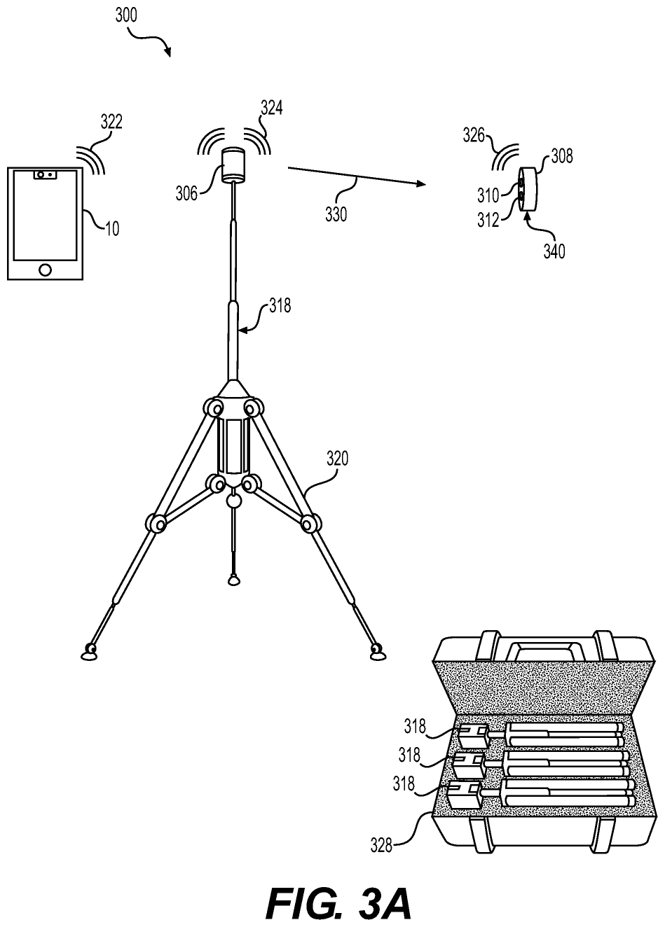

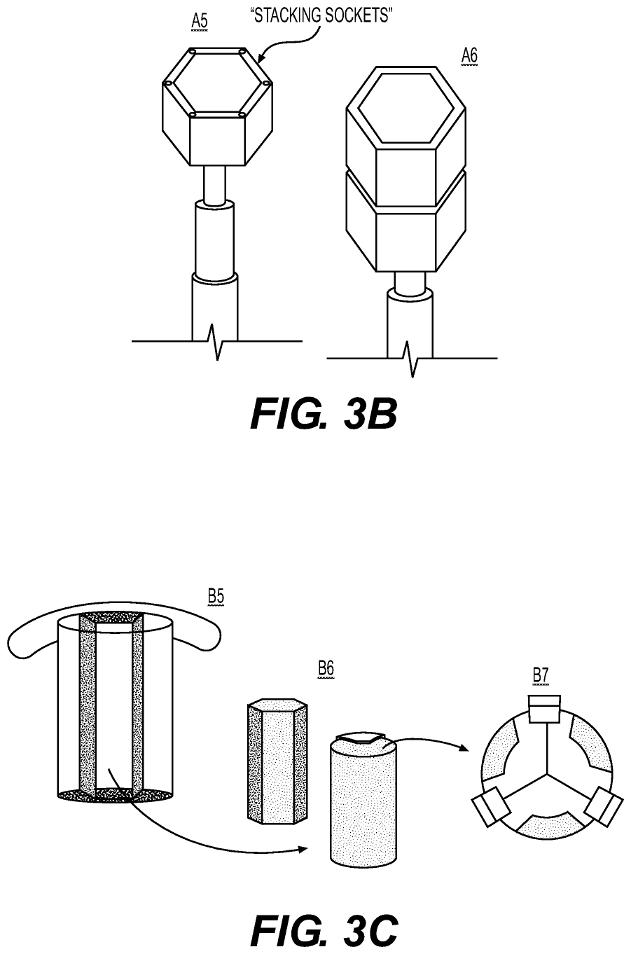

FIG. 3A illustrates an example UV disinfection system 300 having a UV light fixture 318 and a sensor fixture 340. The UV light fixture 318 can direct UV light at and (optionally) wirelessly communicate to the sensor fixture 340 through a line of sight path 330. A UV light source 306 can be carried by the UV light fixture 318. The UV light source 306 can have similar functionality as the UV light sources 106, 206 illustrated in FIGS. 2 and 3. FIG. 3B is an illustration of example UV light emitters usable with the UV light source 306 illustrated in FIG. 3A or any of the other UV light sources described herein. FIG. 3B can include a cartridge that is modular, and the UV light source 306 can be configured to accommodate a variable number of such modular cartridges and/or cartridges of various UV output capabilities, thereby facilitating customization of the UV light source output capabilities. FIG. 3C is an illustration of an example rechargeable battery usable with the example UV disinfection system to provide power to the UV light fixtures 318. FIG. 3C can also be modular, and the UV light fixture 318 can be configured to accommodate a variable number of such modular batteries and/or modular batteries of various capacity, thereby facilitating swapping of rechargeable batteries and/or customization of battery life. A UV light detector 308 can be carried by the sensor fixture 340. The UV light detector 308 can have similar functionality as the UV light detectors 108, 208 illustrated in FIGS. 1 and 2 and can include a 222 nm sensor 310 and a 254 nm sensor 312 functioning similarly to the corresponding sensors 110, 112, 210, 212 illustrated in FIGS. 1 and 2.

The UV light fixture 318 can carry a distance sensor and/or motion sensor having functionality similar to the corresponding components 104, 118, 204, 218 illustrated in FIGS. 1 and 2. Additionally, or alternatively, the sensor fixture 340 can carry a distance sensor and/or motion sensor having functionality similar to the corresponding components 104, 118, 204, 218 illustrated in FIGS. 1 and 2. Preferably the UV light fixture 318 is equipped with a user interface having functionality similar to the user interfaces 102, 202 illustrated in FIGS. 1 and 2. Additionally, or alternatively, the sensor fixture 340 can be equipped with a user interface having functionality similar to the user interfaces 102, 202 illustrated in FIGS. 1 and 2.

As illustrated, the light fixture 318 includes a tripod 320. The tripod 320 can be collapsible to be transported with additional light fixtures 318 in a luggage 328. The luggage 328 can be configured similar to ruggedized luggage suitable for transportation of scientific tools and/or electrical equipment. The light fixture 318 is configured to be portable so that it can be transported to spaces for disinfection. The light fixture 318 can be configured in a multitude of other form factors including portable structures, self-propelled structures (e.g. automated vehicle, drone, etc.), non-portable electrical installations, and the like as understood by a person skilled in the pertinent art.

The sensor fixture 340 can have a multitude of form factors including portable structures, non-portable installations, or semi-portable (e.g. designed for long-term stick-on without modifying the surface to which it is attached and easy removal without damaging the surface to which it is attached).

As illustrated, the external computing system 10, the UV light fixture 318, and the sensor fixture 340 can each respectively be equipped with wireless communication capabilities 322, 324, 326 (e.g. receiver, transmitter, or transceiver as appropriate).

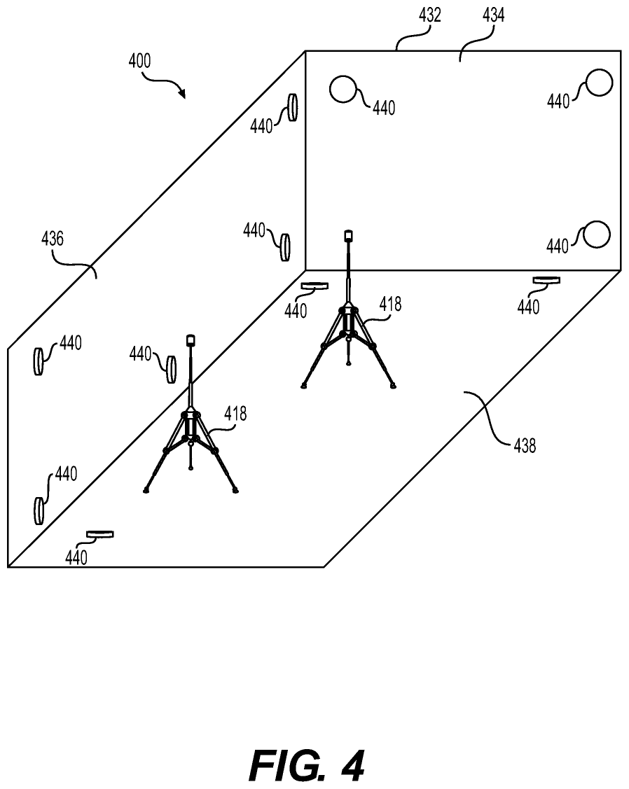

FIG. 4 illustrates another example UV disinfection system 400 configured to disinfect a space 432 which is sized to be occupied by people and/or animals. The space 432 can be as small as a closet or personal office, as large as a C-17 cargo bay, or beyond. FIG. 4 illustrates two portable UV light fixtures 418 configured similarly to the UV light fixture 318 illustrated in FIG. 3A and multiple sensor fixtures 440 configured similarly to the portable sensor fixtures 340 illustrated in FIG. 3A. The sensor fixtures 440 can be portable, or alternatively be installed in the space 432, which can be advantageous if the space 432 is routinely disinfected. The illustrated system 400 can include more or fewer portable UV light fixtures 418 and sensor fixtures 440 to achieve desired dosage in a desired amount of time based on the size and configuration of the space 432. The UV light fixtures 418 can be adjustable in height from the floor 438, in some examples up to about 15 feet from the floor 438.

An example process for disinfecting the space 432 can proceed as follows. One or more of the portable UV light fixtures 418 can be placed in the space 432. If the UV light fixtures are equipped with a distance sensor such as the distance sensors 118, 218 illustrated in FIGS. 1 and 2, the distance sensor can be used to determine the relative position of the UV light fixtures 418 to walls 434, 436 of the space 432 and/or other target surfaces of the space 432. In some examples, the relative position can be used to determine a viable placement of UV light fixtures 418 to achieve a desired dosage within a desired timeframe. For instance, the UV light fixtures 418 can be in communication with an external system 10 running an application that can receive the relative position of the UV light fixtures 418 to the walls 434, 436 and provide instructions to relocate the UV light fixtures 418 to more optimal position. As another example, the UV light fixtures 418 can be configured to provide the instructions for relocation. The instructions can be configured to instruct a human operator to reposition the UV light fixtures 418 for instance by displaying a map or providing other audible, visual, and/or tactile output. Alternatively, although not illustrated, the portable UV light fixtures 418 can be capable of moving themselves to more optimal positions, in which case the instructions for relocation can be carried by electrical signals and/or can be computer readable. Regardless of the form of the instructions or the method of relocation, the UV light fixtures can be moved to final positions based at least in part on data from the distance sensors. In their final positions, the distance sensors can again be used to determine relative positions of the UV light fixtures 418 to the walls 434, 436. Given a known intensity from the UV light source, disinfection time can be calculated based on the final positions of the UV light fixtures 418 in relation to the walls 434, 436. In some examples, intensity of UV light output can be adjusted based on desired dosage and desired disinfection timeframe.

UV light sources of the UV light fixtures 418 can be activated to begin disinfection (e.g. via an activation input 103, 203 such as illustrated in FIGS. 1 and 2). UV light detectors of the sensor fixtures 440 can transmit electrical signals indicating exposure to UV light. In some examples, the system 400 can be configured to calculate dosage at each of the sensor fixtures 440 and compare that dosage to a predetermine threshold. The system 400 can be configured to cease illumination of the UV light source when dosage at all, or some acceptable percentage of the sensor fixtures 440 surpass the predetermined threshold. Actual disinfection time can be compared to calculated disinfection time as a system functionality check. Alternatively, the system 400 can be configured to cease illumination of the UV light source following elapse of the disinfection time, in which case the sensors can be used simply as a binary check to ensure illumination from each wavelength occurred, or dosage at each sensor fixture 440 can be measured and compared to predetermined threshold(s) as a system check.

In example systems 400 including a distance sensor, the sensor fixtures 440 can be used to verify that the UV light sources are functioning as expected, and the system 400 can be configured to cease illumination of the UV light source when the calculated disinfection time has elapsed, where the disinfection time is calculated based on the final positions of the UV light fixtures 418 in relation to the walls 434, 436. In some examples, system 400 can determine the relative position of the sensor fixtures 440 to the UV light fixtures 418. In examples systems 400 including a distance sensor and where the relative positions of the sensor fixtures 440 to the UV light fixtures 418 are known, the system 400 can determine the predetermined threshold individually for a sensor fixture 440 based on its position in relation to the UV light fixtures 418 in comparison to the UV light fixture's distance from target surfaces. In other words, a sensor fixture 440 positioned to receive a larger dose than the furthest reaches of the target surfaces can have a higher predetermined threshold so that a desired dosage can be achieved at the furthest reaches of the target surfaces.

In example systems 400 including a motion sensor, the system 400 can be configured to prevent illumination of the UV light source at potentially harmful (to humans and/or animals) wavelengths when motion is detected by the motion sensor. In some examples, the system 400 can be configured to allow illumination of the UV light source at less harmful or non-harmful wavelengths when motion is detected by the motion sensor. Additionally, or alternatively, the system 400 can be configured to cease all illumination of UV light when motion is detected by the motion sensor.

The system 400 can be adaptable to meet different end user's disinfection needs. In some examples, UV power output can be adjusted. As illustrated, spatial placement of UV light fixtures 418 and sensor fixtures 440 can be customized. Desired dose can be selected based on pathogen and/or degree of disinfection. Such configurability can provide robust disinfection of large areas for various sectors including transportation services (e.g. commercial airlines, military patient transport aircraft, buses, or trains), hospitals, hotels, schools, airports, churches restaurants, and agriculture processing plants (produce harvesting, meat processing and packaging). For instance, the commercial airline industry has over 500 airports in the US alone, and 17,000 airports worldwide operating over 100 thousand flights per day. By adjusting UV output and number of UV light fixtures 418 and sensor fixtures 440, the system 400 can be scaled down to disinfect a passenger cabin of a small aircraft and scaled up to disinfect an entire airport. Hospitals and clinics have over 400 million square meters of patient rooms and sterile surgical spaces. The system 400 can therefore be utilized by a multitude of industries worldwide that need to employ daily disinfection procedures.

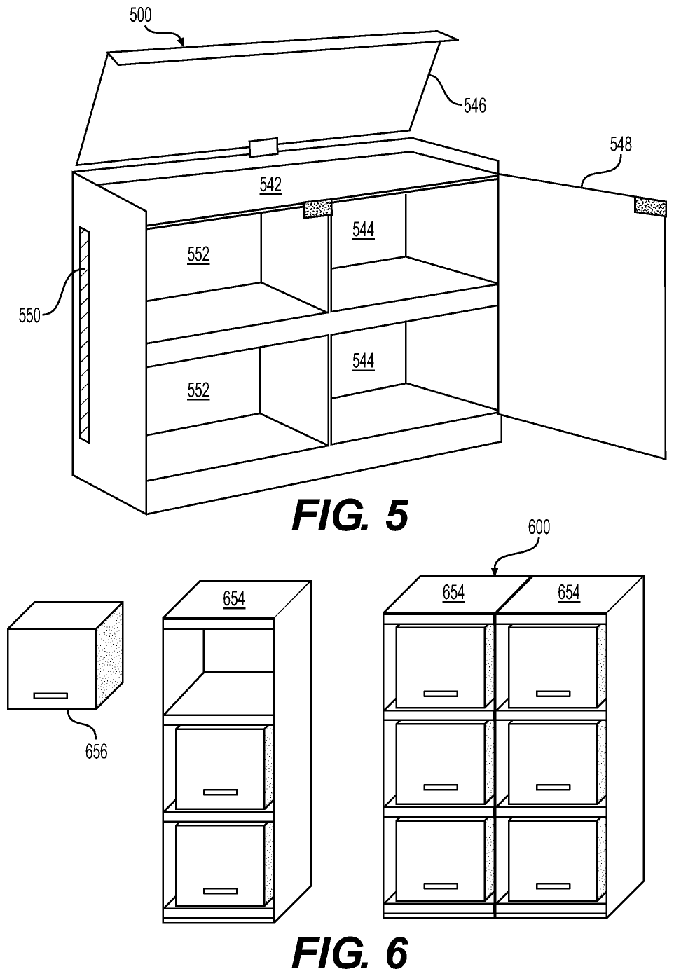

FIG. 5 is an illustration of another example UV disinfection system 500 including disinfection compartments 542, 544. The system 500 can include UV light sources configured similarly to the UV light sources 106, 206 illustrated in FIGS. 1 and 2. The UV light sources can be positioned to illuminate the disinfection compartments 542, 544. Each of the compartments 542 can have a respective door 546, 548 that can be opened so that objects such as medical equipment, computer equipment, personal protective equipment, office supplies, etc. can be inserted into the compartment 542, 544. When the respective door 546, 548 is closed, light and objects can be inhibited from exiting the compartment.

The UV disinfection system 500 can include UV light detectors configured similarly to the UV light detectors 108, 208 illustrated in FIGS. 1 and 2. The UV light detectors can be positioned within the disinfection compartments 542, 544. The UV disinfection system can be configured to cease illumination of the UV light source of a compartment when exposure of UV light to the UV light detector in that compartment 542, 544 reaches a predetermined threshold. The predetermined threshold can be based on dosages effective to disinfect one or more target microbes. The predetermined threshold can otherwise be determined as disclosed elsewhere herein. Alternatively, the system 500 can be configured to detect the presence or absence of illumination from the UV light source in the same compartment, as a binary check (e.g. functional or non-functional) of the functionality of the UV light source. The UV light source for each compartment 542, 544 can include a modular cartridge so that the UV light source can be readily replaced when needed. The system 500 can further be configured to accept a variable number of UV light source cartridges and/or cartridges of various output ratings so that UV power within some or all of the compartments 542, 544 is customizable.

The system 500 can be configured as a semi-stationary piece of office furniture, similar to a desk, filing cabinet, etc. or configured to greater portability, e.g. additional of wheels. The cabinet can be powered by a cord with plug, hardwired junction, and/or rechargeable battery.

The UV disinfection system 500 can include a user interface configured similarly to the user interface 102, 202 illustrated in FIGS. 1 and 2. The user interface can include an activation input configured similarly to the activation input 103 illustrated in FIG. 1. The user interface can have buttons or touch screen to allow the user to choose different disinfection programs. The user interface can be configured to display the activity/status of the program in real time. The activation input can be configured to activate to cause the UV light source to illumination for a respective compartment 542, 544 when the respective door 546, 548 of that compartment closes. The system 500 can include a processor and memory configured similarly to the processor 214 and memory 216 illustrated in FIG. 2. The memory can include instructions that cause the processor to detect status of each door 546, 548 as open or closed. When the door is closed, the processor can provide a control signal to the UV light source to cause the UV light source to illuminate. When the door is open, the processor can provide a control signal to the UV light source to prevent or cease illumination of the UV light source at least at 254 nm or other wavelengths that might present a health risk.

The UV disinfection system 500 can further include storage compartments 552. The storage compartments can be adjustable to accommodate different sizes of items.

The UV disinfection system 500 can further include an air circulation system 550. The air circulation system 550 can be configured to cool the UV light source and other mechanical or electrical components of the UV disinfection system 500 that may generate heat during operation. The air circulation system 550 can further include a UV light chamber which is continuously illuminated with UV light, while air is circulated, to disinfect air.

The UV disinfection system 500 can have a form factor similar to that of a filing cabinet so that it can be readily placed into an office, a classroom, a hospital room, etc. to provide disinfection of objects and air.

The UV disinfection system 500 can include an additional UV light source configured to illuminate a space outside of the system 500 similar to the configuration of the UV light source 306 of the system 300 illustrated in FIG. 3A. For instance, the system 500 can include one or more cabinets having a similar form factor as illustrated that are positioned within a room such as an office, classroom, hospital room, etc., and the additional UV light source of each cabinet can be configured to illuminate according to a process similar to as described in relation to FIG. 4 when the space is unoccupied (e.g. office occupant out to lunch, children of classroom at recess, etc.).

FIG. 6 is an illustration of another example UV disinfection system 600 including modular disinfection compartments 656 and a modular shelving system 654. The compartments 656 can include a UV light source and a UV light detector configured similarly to the UV light source 106, 206 and UV light detector 108, 208 illustrated in FIGS. 1 and 2. The compartment 656 can be closable to prevent escape of UV light either via a lid or closable opening of the compartment 656 itself or via fitment into the shelving system 654. The shelving system 654 can include towers 654 that can be positioned together similar to modular storage cube furniture, and the disinfection compartments 656 can be placed into the shelving system 654 similar to storage cubes into modular storage cube furniture. Objects can be placed into the compartments 656 and disinfected in a process similar to disinfection in UV compartments 542, 544 illustrated in FIG. 5. In some examples, each disinfection compartment 656 can be configured to function independent of the modular shelving system 654, having its own power supply and/or power cord connection and system components such as in the systems 100, 200 illustrated in FIGS. 1 and 2. Additionally, or alternatively, the shelving system 654 can provide power to the disinfection compartments, a user interface, processor, UV light source, and/or UV light detector. In some examples, each compartment 656 can be separately configurable for a prescribed intensity of UV light, duration of UV light, and/or wavelength of UV light. Such configurations can be based on a degree of disinfection/sterilization of one or more target microorganisms. The shelving system 654 can further include an air circulation system configured to cool the compartments 656 and/or disinfect air similar to the air circulation system 550 illustrated in FIG. 5. The air circulation system can be positioned to flow air through the back of the shelving system 654 and/or through the bottom of the shelving system 654.

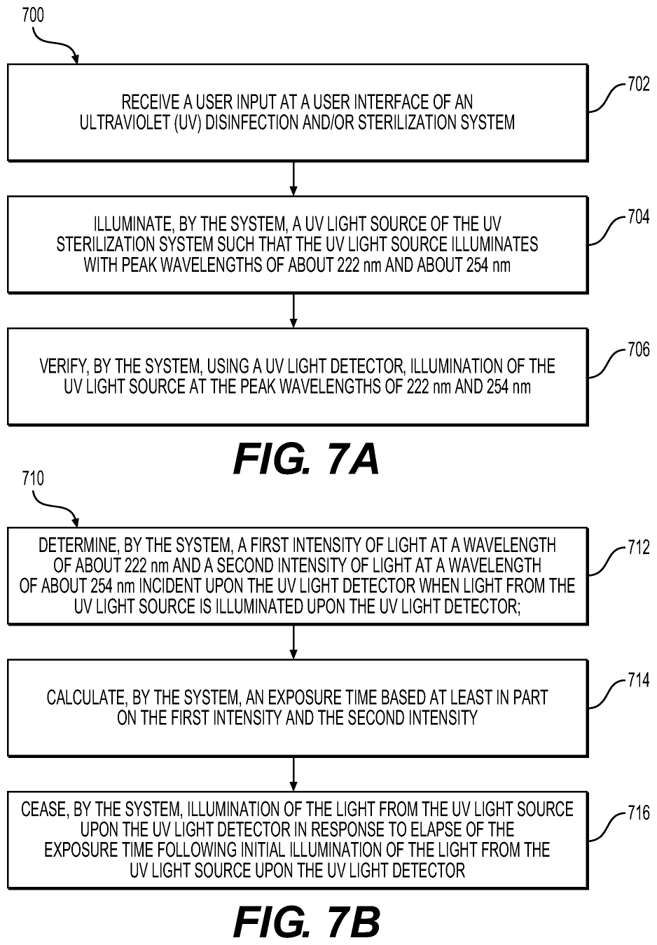

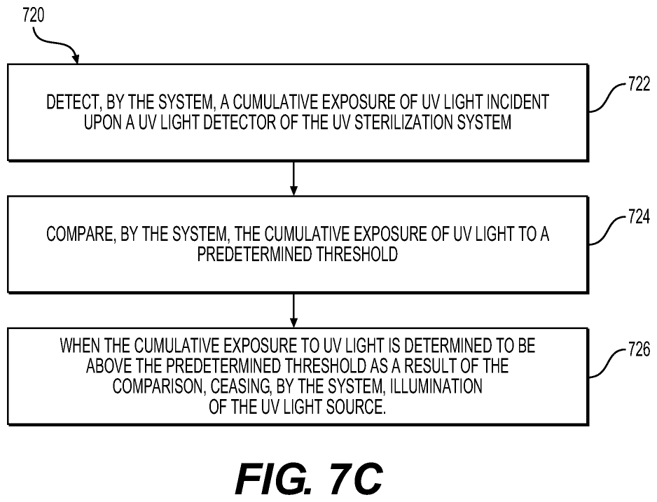

FIGS. 7A through 7C are flow diagrams illustrating methods 700, 710, 720 of UV disinfection. A decontamination process can include a combination of steps from the methods 700, 710, 720. Steps are presented in no particular order. A decontamination process can include additional steps as would be appreciated and understood by a person of ordinary skill in the art. The example methods can be performed by an example system 100, 200, 300, 400, 500, 600 as disclosed herein, a variation thereof, or an alternative thereto as would be appreciated and understood by a person of skilled in the pertinent art.

At step 702 of the method 700 illustrated in FIG. 7A, the user interface of a UV disinfection and/or sterilization system (referred to hereafter as “UV disinfection system” for simplicity) can receive a user input. The user interface can be configured similarly to any one of the user interfaces 102, 202 illustrated or described herein, a variation thereof, or an alternative thereto as would be appreciated and understood by a person of skilled in the pertinent art.

At step 704, the UV disinfection system can illuminate a UV light source of the UV disinfection system. The UV light source can be configured similarly to any one of the UV light sources 106, 206, 306 illustrated or described herein, a variation thereof, or an alternative thereto as would be appreciated and understood by a person of skilled in the pertinent art. The UV light source can illuminate with peak wavelengths of about 222 nm and about 254 nm.

At step 706, the UV disinfection system can verify illumination of the UV light source at the peak wavelengths of 222 nm and 254 nm. Verification of the illumination can be achieved using the UV light detector.

Following the method 700 outlined in FIG. 7A, a disinfection process can proceed by performing the method 710 illustrated in FIG. 7B, the method 720 illustrated in FIG. 7C, and/or a process including steps from both methods 710, 720 illustrated in FIGS. 7B and 7C.

At step 712 of method 710 illustrated in FIG. 7B, the UV disinfection system can determine a first intensity of light at a wavelength of about 222 nm and a second intensity of light at a wavelength of about 254 nm incident upon the UV light detector when the UV light source is illuminated upon the UV light detector.

At step 714, the UV disinfection system can calculate an exposure time based at least in part on the first intensity and the second intensity determined at step 712.

At step 716, the UV disinfection system can cease illumination of light from the UV light source upon the UV light detector in response to elapse of the exposure time following initial illumination of the light from the UV light source upon the UV light detector.

At step 722 of the method 720 illustrated in FIG. 7C, the UV disinfection system can detect a cumulative exposure of UV light incident upon a UV light detector of the UV disinfection system. The UV light detector can be configured similarly to any one of the UV light detectors 108, 208, 308 illustrated or described herein, a variation thereof, or an alternative thereto as would be appreciated and understood by a person of skilled in the pertinent art.

At step 724, the UV disinfection system can compare the cumulative exposure of UV light to a predetermined threshold. The comparison can be carried out by a processor of the system such as the processor 214 illustrated in FIG. 2.

At step 726, the UV disinfection system can cease illumination of the UV light source when the cumulative exposure to UV light is determined to be above the predetermined threshold based on the comparison at step 724.

One example decontamination process can include steps 712 and 714 from method 710 in FIG. 7B and steps 722 and 724 from method 720 in FIG. 7C and either of step 716 from method 710 in FIG. 7B or step 726 from method 720 in FIG. 7C. When the process includes step 716, the comparison of the cumulative exposure to UV light to the predetermined threshold in step 724 can be used to verify expected operation of the UV disinfection system. When the process includes step 726, actual exposure time can be compared to the exposure time calculated in step 714 to verify expected operation of the UV disinfection system.

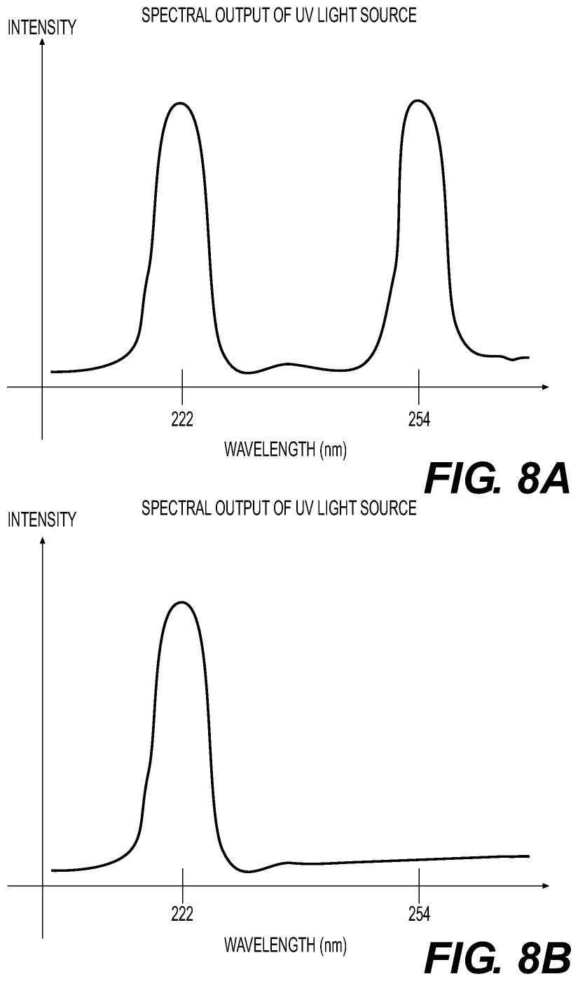

FIGS. 8A and 8B are spectrographs illustrating spectral output of an example UV light source in different modes of operation (where the example UV light source is configured similarly to any of the UV light sources 106, 206, 306 illustrated or otherwise described herein). FIG. 8A illustrates a spectral output of the example UV light source in a mode where there is low risk that illumination will be incident upon people or animals. The UV light source is illuminated with peak wavelengths of about 222 nm and about 254 nm. As illustrated in the spectrograph, it is to be understood, that when discussing illumination at a given peak wavelength, the actual output of the UV light source includes a band of wavelengths including the peak wavelength. FIG. 8B illustrates a spectral output of the example UV light source in a mode where there is a high risk that illumination will be incident upon people or animals. The UV light source is illuminated with a peak wavelength of about 222 nm and light output at 254 nm is substantially reduced or completely eliminated. The peak intensity can also be variable to allow for optimal dosing schemes (i.e. increased UV intensity/power output for increased killing rate but more power/battery usage, or lower UV intensity to conserve power but require longer exposure time).

The descriptions contained herein are examples of embodiments of the invention and are not intended in any way to limit the scope of the invention. As described herein, the invention contemplates many variations and modifications of a disinfection system, including utilizing alternative wavelengths of light, optional functionality (e.g. motion sensing, distance sensing, communication with an external computing system, etc.), configurability for various applications and use cases, materials to be used for the inside of the cabinet walls and shelving to be reflective and/or allow UV light to pass (i.e. fused quartz) to control irradiation trajectory, coating material to prevent degradation of interiors of cabinets, manufacturing designs for mechanical, electrical, and thermal considerations, etc. Modifications that are apparent to those skilled in the art to which this invention pertains are intended to be within the scope of the claims which follow.

What is claimed is:

1. An ultraviolet (UV) disinfection and/or sterilization system comprising:
a UV light detector; and
a UV light source configured to communicate with the UV light detector, the UV light source being configured to:
illuminate, with peak wavelengths of about 222 nm and about 254 nm, and
verify illumination at the peak wavelengths of about 222 nm and about 245 nm based at least in part on UV light detected by the UV light detector.

2. The UV disinfection and/or sterilization system of claim 1, wherein the UV light source is further configured to cease illumination in response to detection, by the UV light detector, of a cumulative exposure to UV light over a predetermine threshold.

3. The UV disinfection and/or sterilization system of claim 1 further comprising:
a plurality of UV light detectors comprising the UV light detector, each of the plurality of UV light detectors configured to detect a respective cumulative exposure to UV light,
wherein the UV light detector is configured to measure a first intensity of light at a wavelength of about 222 nm and a second intensity of light at a wavelength of about 254 nm,
wherein the UV light source is further configured to cease illumination at a calculated time based at least in part on the first intensity of light and the second intensity of light, and
wherein the UV light source is further configured to cease illumination in response to each of the respective cumulative exposure to UV light reaching a predetermined threshold.

4. The UV disinfection and/or sterilization system of claim 1,
wherein the UV light detector comprises a first UV light sensor configured to detect UV light within a first band of wavelengths comprising 222 nm and excluding 254 nm and a second UV light sensor configured to detect UV light within a second band of wavelengths comprising 254 nm and excluding 222 nm, and
wherein the UV light detector is configured to detect a cumulative exposure to UV light such that the cumulative exposure to UV light is based at least in part on UV light detected by the first UV light sensor in the first band of wavelengths and based at least in part on UV light detected by the second UV light sensor in the second band of wavelengths.

5. The UV disinfection and/or sterilization system of claim 1, further comprising:
a user interface configured to communicate with the UV light source and comprising an activation input,
wherein the UV light source is further configured to illuminate with peak wavelengths of about 222 nm and about 254 nm in response to a first activation of the activation input, and
wherein the UV light source is further configured to illuminate without the peak wavelength of about 254 nm in response to a second activation of the activation input.

6. The UV disinfection and/or sterilization system of claim 1, further comprising:
a motion sensor configured to communicate with the UV light source,
wherein the UV light source is further configured to illuminate without the peak wavelength of about 254 nm in response to detection, by the motion sensor, of motion.

7. The UV disinfection and/or sterilization system of claim 1, further comprising:
a compartment comprising a closable opening through which objects can pass when the opening is open and which inhibits light and objects from exiting the compartment when the opening is closed, wherein UV the light source and UV light detector are affixed within the compartment; and
an activation input configured to activate at least in part in response to closing of the closable opening, wherein the UV light source is further configured to illuminate in response to activation of the activation input.

8. The UV disinfection and/or sterilization system of claim 1, further comprising:
a sensor fixture to which the UV light detector is affixed; and
a portable light fixture to which the UV light source is affixed, the portable light fixture being movable in relation to the sensor fixture,
wherein the portable light fixture and the sensor fixture are configured to partially or automatically manage a disinfection operation in a location being disinfected by the UV disinfection system, and
wherein the portable light fixture and/or the sensor fixture are configured to transmit alert messages automatically based on one or more predetermined disinfection parameters of the location being disinfected, the alert messages comprising an indication of disinfection and caused to be manually or automatically transmitted to a user computing device or other user computing devices securely connected therewith so that end-users can quickly and reliably detect that the location is disinfected.

9. The UV disinfection and/or sterilization system of claim 1,
wherein the UV light source comprises a distance sensor configured to measure a distance from a surface in line of sight of UV light from the UV light source.

10. The UV disinfection and/or sterilization system of claim 1 being configured to disinfect surfaces and circulate air to disinfect the circulated air.

11. An ultraviolet (UV) disinfection and/or sterilization system comprising:
a UV light detector;
a UV light source configured to illuminate with peak wavelengths of about 222 nm and about 254 nm;
a processor in communication the UV light detector and the UV light source; and
non-transitory computer readable medium in communication with the processor and comprising instructions thereon, that when executed by the processor, cause the processor to:
provide, in response to receiving an activation signal, a first control signal to the UV light source to cause the UV light source to illuminate,
receive a detector signal from the UV light detector, and
verify, based at least in part on the detector signal, illumination of the UV light source at the peak wavelengths of 222 nm and 254 nm.

12. The UV disinfection and/or sterilization system of claim 11, wherein the non-transitory computer readable medium further comprises instructions thereon, that when executed by the processor, cause the processor to:
determine, based at least in part on the detector signal, a cumulative exposure to UV light,
compare the cumulative exposure to UV light to a predetermined threshold, and
when the cumulative exposure to UV light is determined to be above the predetermined threshold as a result of the comparison, provide, a second control signal to the UV light source to cause the UV light source to cease illumination.

13. The UV disinfection and/or sterilization system of claim 11, wherein the non-transitory computer readable medium further comprises instructions thereon, that when executed by the processor, cause the processor to:
control intensity of UV light output from the UV light source based at least in part on the detector signal.

14. The UV disinfection and/or sterilization system of claim 11, wherein the non-transitory computer readable medium further comprises instructions thereon, that when executed by the processor, cause the processor to:
determine, based at least in part on the detector signal, a first intensity of light at a wavelength of about 222 nm and a second intensity of light at a wavelength of about 254 nm,
calculate, based at least in part on the first intensity and the second intensity, an exposure time, and
provide, in response to elapse of the exposure time following the providing of the first control signal, a second control signal to the UV light source to cause the UV light source to cease illumination.

15. The UV disinfection and/or sterilization system of claim 11, further comprising:
a user interface in communication with the processor and configured to provide the activation signal to the processor in response to a user input,
wherein the non-transitory computer readable medium further comprises instructions thereon, that when executed by the processor, cause the processor to:
receive a wavelength selection signal from the user interface, and
provide, based at least in part on the wavelength selection signal, a third control signal to the UV light source to cause the UV light source to illuminate without the peak wavelength of about 254 nm, and
wherein the user interface comprises a wireless transceiver configured to:
wirelessly receive a user input from a computing device that is separate and distinct from the UV light detector and the UV light source, and
generate the activation signal based at least in part on the user input.

16. The UV disinfection and/or sterilization system of claim 11, wherein the UV light detector comprises:
a first UV light sensor configured to detect UV light within a first band of wavelengths comprising 222 nm and excluding 254 nm, and
a second UV light sensor configured to detect UV light within a second band of wavelengths comprising 254 nm and excluding 222 nm,
wherein the non-transitory computer readable medium further comprises instructions thereon, that when executed by the processor, cause the processor to:
receive a first sensor signal from the first UV light sensor,
receive a second sensor signal from the second UV light sensor, and
calculate a cumulative exposure to UV light based at least in part on the first sensor signal and the second sensor signal.

17. The UV disinfection and/or sterilization system of claim 11, further comprising:
a motion sensor configured to communicate with the processor,
wherein the non-transitory computer readable medium further comprises instructions thereon, that when executed by the processor, cause the processor to:
receive a motion sense signal from the motion sensor, and
provide, based at least in part on the motion sense signal, a fourth control signal to the UV light source to cause the UV light to illuminate without the peak wavelength of about 254 nm.

18. The UV disinfection and/or sterilization system of claim 11, further comprising:
a compartment comprising a closable opening through which objects can pass when the opening is open and which inhibits light and objects from exiting the compartment when the opening is closed,
wherein the light source and light sensor are affixed within the compartment, and
wherein the non-transitory computer readable medium further comprises instructions thereon, that when executed by the processor, cause the processor to:
detect status of closable opening as open or closed, and
when the status of the closable opening is open, provide a fifth control signal to the UV light source to inhibit the UV light source from illuminating at the peak wavelength of about 254 nm.

19. The UV disinfection and/or sterilization system of claim 11, further comprising:
a plurality of UV light detectors comprising the UV light detector, each of the plurality of UV light detectors configured to provide, to the processor, a respective detector signal,
wherein the non-transitory computer readable medium further comprises instructions thereon, that when executed by the processor, cause the processor to:
receive the respective detector signals,
determine a respective cumulative exposure to UV light for each of the respective detector signals,
compare each of the respective cumulative exposure to UV light to a predetermined threshold, and
when each of the respective cumulative exposure to UV light are determined to be above the predetermined threshold as a result of the comparison, provide, a second control signal to the UV light source to cause the UV light source to cease illumination.

20. The UV disinfection and/or sterilization system of claim 11,
wherein the UV light source comprises a distance sensor configured to measure a distance from a surface in line of sight of UV light from the UV light source.
